(19) United States
(12) Patent Application Publication
(10) Pub. No.: US 2015/0075062 A1
(43) Pub. Date: Mar. 19, 2015

(54) ALCOHOL COMPOSITIONS AND A PROCESS FOR THEIR PRODUCTION

(71) Applicant: INEOS BIO SA, Rolle (CH)

(72) Inventors: Peter Simpson Bell, Dunblane (GB); Michael Sean Slape, Naperville, IL (US); Scot Porento, Channahon, IL (US)

(73) Assignee: INEOS BIO SA, Rolle (CH)

(21) Appl. No.: 14/482,356

(22) Filed: Sep. 10, 2014

Related U.S. Application Data
(60) Provisional application No. 61/877,529, filed on Sep. 13, 2013.

(51) Int. Cl. C10L 1/182; C12P 7/06; C10L 1/19; C10L 1/188

(52) U.S. Cl. CPC C10L 1/1824; C10L 1/188; C12P 7/06; C10L 1/19
USPC 44/388; 44/451; 44/411; 435/161

(57) ABSTRACT

An alcohol product composition is provided that may be used directly for blending with existing fuel sources. More specifically, the alcohol product composition includes ethanol and organic compositions which act as a denaturant. Further, a process for production of ethanol compositions is provided that includes providing a permeate to a distillation tower, removing an ethanol draw-off composition from the distillation tower, removing a side draw from the distillation tower to provide side-draw composition, combining the ethanol draw-off composition and side-draw composition to provide an alcohol composition

This application claims the benefit of U.S. Provisional Application No. 61/877,529, filed Sep. 13, 2014, which is incorporated in its entirety herein by reference.

An alcohol product composition is provided that may be used directly for blending with existing fuel sources. More specifically, the alcohol product composition includes ethanol and organic compositions which act as a denaturant. Further, a process for production of ethanol compositions is provided that includes providing a permeate to a distillation tower, removing an ethanol draw-off composition from the distillation tower, removing a side draw from the distillation tower to provide side-draw composition, combining the ethanol draw-off composition and side-draw composition to provide an alcohol stream that is then sent to a dehydration unit, providing a finished alcohol composition.

### BACKGROUND

Alcohols such as ethanol for industrial use are conventionally produced from petrochemical feed stocks, such as oil, natural gas, or coal, from feed stock intermediates, such as syngas, or from starchy materials or cellulose materials, such as corn or sugar cane. Conventional methods for producing ethanol from petrochemical feed stocks, as well as from cellulose materials, include the acid-catalyzed hydration of ethylene, methanol homologation, direct alcohol synthesis, and Fischer-Tropsch synthesis. Instability in petrochemical feed stock prices contributes to fluctuations in the cost of conventionally produced ethanol, making the need for alternative sources of ethanol production all the greater when feed stock prices rise. Starchy materials, as well as cellulose material, are converted to ethanol by fermentation. However, fermentation is typically used for consumer production of ethanol for fuels or consumption. In addition, fermentation of starchy or cellulose materials competes with food sources and places restraints on the amount of ethanol that can be produced for industrial use. Conventional ethanol compositions formed as a result of the above-identified processes may contain impurities, such as for example organic acids, which must be removed.

Acetogenic microorganisms can produce alcohol from carbon monoxide (CO) through fermentation of gaseous substrates. Fermentations using anaerobic microorganisms from the genus Clostridium produce ethanol and other useful products. For example, U.S. Pat. No. 5,173,429 describes Clostridium ljungdahlii ATCC No. 49587, an anaerobic microorganism that produces ethanol and acetate from synthesis gas. U.S. Pat. No. 5,807,722 describes a process and apparatus for converting waste gases into organic acids and alcohols using Clostridium ljungdahlii ATCC No. 55380. U.S. Pat. No. 6,136,577 describes a process and apparatus for converting waste gases into ethanol using Clostridium ljungdahlii ATCC No. 55988 and 55989.

Ethanol composition produced for transport fuel uses are required to be denatured by blending with gasoline or other approved denaturants. Denaturing ethanol requires additional capital costs in terms of equipment and product supply. Further, the cost of denaturants may vary resulting in increased product costs.

### SUMMARY

An alcohol product composition is provided that may be used directly for blending with existing fuel sources. The present alcohol product composition does not require further blending with gasoline denaturant as the alcohol product composition contains organic compositions which act and qualify as a denaturant. The organic compositions are formed during the fermentation process and are maintained in the alcohol product composition provided from dehydration. The present fermentation, distillation and dehydration processes are effective for providing an alcohol product composition that does not require further processing to remove impurities.

An alcohol product composition includes about 92 weight percent or more ethanol, about 0.5 to about 8 weight percent organic composition, about 1 weight percent or less water, and about 0.5 weight percent or less organic acid. In one aspect, the organic composition is selected from the group consisting of n-butanol, isobutanol, pentanol, hexanol, propanol, methanol, ethyl acetate, fatty acids, esters and mixtures thereof. The organic composition may be substantially n-butanol. In another aspect, the organic composition may further include one or more hydrocarbons, such as for example, gasoline. Organic acid may include organic acid selected from the group consisting of acetic acid, butyric acid, maleic acid, malonic acid, malic acid, propionic acid, succinic acid, oxalic acid, lactic acid, fumaric acid, glutaric acid, formic acid, citric acid, uric acid, and mixtures thereof.

In another aspect, a process for producing an alcohol product composition includes providing a permeate to a distillation tower, removing an ethanol draw-off composition from the distillation tower, removing a side draw from the distillation tower to provide side-draw composition, combining the ethanol draw-off composition and side-draw composition to provide an alcohol composition having more than 1 weight percent water, and dehydrating the alcohol composition to provide an alcohol product composition having at about 92 weight % or more ethanol, about 1 weight % or less water, about 0.5 to about 8 weight percent organic composition, and about 0.5 weight percent or less organic acid. In this aspect, the permeate includes from about 1 to about 5 weight percent ethanol and the permeate is provided from a fermentation of a CO-containing gaseous substrate.

In accordance with the process, the organic composition may includes an organic composition selected from the group consisting of n-butanol, isobutanol, pentanol, hexanol, propanol, methanol, ethyl acetate, fatty acids, esters and mixtures thereof. In one aspect, the organic composition is n-butanol. In this aspect, the ethanol product composition includes about 0.5 weight percent to about 6 weight percent n-butanol. Organic acid may include organic acid selected from the group consisting of acetic acid, butyric acid, maleic acid, malonic acid, malic acid, propionic acid, succinic acid, oxalic acid, lactic acid, fumaric acid, glutaric acid, formic acid, citric acid, uric acid, and mixtures thereof.

In another aspect, a bioethanol product includes about 92 weight percent or more ethanol, about 0.5 to about 8 weight percent organic composition, about 1 weight % or less water, and about 0.5 weight percent or less organic acid. The bioethanol product is produced by a process that includes fermenting a CO-containing substrate and separating a permeate from the fermentation, providing the permeate to a distillation tower, removing an ethanol draw-off composition from the distillation tower, removing a side draw from the distillation tower to provide side-draw composition, and combining the ethanol draw-off composition and side-draw composition to provide an alcohol composition having more than 1 weight percent water; and dehydrating the alcohol composition to provide the bioethanol product.

In another aspect, a process for producing an alcohol product composition includes fermenting a CO-containing gaseous substrate to produce a first alcohol composition, purifying part or all of the first alcohol composition to produce a second alcohol product composition having about 92 weight percent or more ethanol; about 0.5 to about 8 weight percent organic composition; about 1 weight % or less water; and about 0.5 weight percent or less organic acid. In this aspect, purifying is selected from the group consisting of dehydration, filtration and mixtures thereof. The first alcohol composition includes from about 1 to about 5 weight percent ethanol and may be provided from a fermentation of a CO-containing gaseous substrate. The fermentation is effective for providing a STY of at least about 10 g total alcohol/(L·day), and in another aspect, at least about 10 g ethanol/(L·day).

### BRIEF DESCRIPTION OF FIGURES

The above and other aspects, features and advantages of several aspects of the process will be more apparent from the following figures.

FIG. 1 illustrates a process and system for fermentation of syngas and production of an alcohol product.

FIG. 2 generally illustrates a distillation process.

FIG. 3 illustrates distillation column operation.

Corresponding reference characters indicate corresponding components throughout the several views of the drawings. Skilled artisans will appreciate that elements in the figures are illustrated for simplicity and clarity and have not necessarily been drawn to scale. For example, the dimensions of some of the elements in the figures may be exaggerated relative to other elements to help to improve understanding of various aspects. Also, common but well-understood elements that are useful or necessary in a commercially feasible aspect are often not depicted in order to facilitate a less obstructed view of these various aspects.

### DETAILED DESCRIPTION

The following description is not to be taken in a limiting sense, but is made merely for the purpose of describing the general principles of exemplary embodiments. The scope of the invention should be determined with reference to the claims.

### DEFINITIONS

Unless otherwise defined, the following terms as used throughout this specification for the present disclosure are defined as follows and can include either the singular or plural forms of definitions below defined:

The term “about” modifying any amount refers to the variation in that amount encountered in real world conditions, e.g., in the lab, pilot plant, or production facility. For example, an amount of an ingredient or measurement employed in a mixture or quantity when modified by “about” includes the variation and degree of care typically employed in measuring in an experimental condition in production plant or lab. For example, the amount of a component of a product when modified by “about” includes the variation between batches in a multiple experiments in the plant or lab and the variation inherent in the analytical method. Whether or not modified by “about,” the amounts include equivalents to those amounts. Any quantity stated herein and modified by “about” can also be employed in the present disclosure as the amount not modified by “about”.

The term “gaseous substrate” is used in a non-limiting sense to include substrates containing or derived from one or more gases.

The term “syngas” or “synthesis gas” means synthesis gas which is the name given to a gas mixture that contains varying amounts of carbon monoxide and hydrogen. Examples of production methods include steam reforming of natural gas or hydrocarbons to produce hydrogen, the gasification of coal and in some types of waste-to-energy gasification facilities. The name comes from their use as intermediates in creating synthetic natural gas (SNG) and for producing ammonia or methanol. Syngas is combustible and is often used as a fuel source or as an intermediate for the production of other chemicals.

The term “fermentor” includes a fermentation device consisting of one or more vessels and/or towers or piping arrangements, which includes the Continuous Stirred Tank Reactor (CSTR), Immobilized Cell Reactor (ICR), Trickle Bed Reactor (TBR), Moving Bed Biofilm Reactor (MBBR), Bubble Column, Gas Lift Fermenter, Membrane Reactor such as Hollow Fibre Membrane Bioreactor (HFMBR), Static Mixer, or other vessel or other device suitable for gas-liquid contact.

The terms “fermentation”, fermentation process” or “fermentation reaction” and the like are intended to encompass both the growth phase and product biosynthesis phase of the process. In one aspect, fermentation refers to conversion of CO to alcohol.

The term “cell density” means mass of microorganism cells per unit volume of fermentation broth, for example, grams/liter.

The term “cell recycle” refers to separation of microbial cells from a fermentation broth and returning all or part of those separated microbial cells back to the fermentor. Generally, a filtration device is used to accomplish separations.

As used herein “gasoline” is defined per 40 CFR 80.2, which is incorporated herein by reference.

### CO-Containing Substrate

A CO-containing substrate may include any gas that includes CO. In this aspect, a CO-containing gas may include syngas, industrial gases, and mixtures thereof.

Syngas may be provided from any know source. In one aspect, syngas may be sourced from gasification of carbonaceous materials. Gasification involves partial combustion of biomass in a restricted supply of oxygen. The resultant gas mainly includes CO and H₂. In this aspect, syngas will contain at least about 10 mole % CO, in one aspect, at least about 20 mole %, in one aspect, about 10 to about 100 mole %, in another aspect, about 20 to about 100 mole % CO, in another aspect, about 30 to about 90 mole % CO, in another aspect, about 40 to about 80 mole % CO, and in another aspect, about 50 to about 70 mole % CO. Some examples of suitable gasification methods and apparatus are provided in U.S. Ser. Nos. 61/516,667, 61/516,704 and 61/516,646, all of which were filed on Apr. 6, 2011, and in U.S. Ser. Nos. 13/427,144, 13/427,193 and 13/427,247, all of which were filed on Mar. 22, 2012, and all of which are incorporated herein by reference.

In another aspect, the process has applicability to supporting the production of alcohol from gaseous substrates such as high volume CO-containing industrial flue gases. In some aspects, a gas that includes CO is derived from carbon containing waste, for example, industrial waste gases or from the gasification of other wastes. As such, the processes represent effective processes for capturing carbon that would otherwise be exhausted into the environment. Examples of industrial flue gases include gases produced during ferrous metal products manufacturing, non-ferrous products manufacturing, petroleum refining processes, gasification of coal, gasification of biomass, electric power production, carbon black production, ammonia production, methanol production and coke manufacturing.

Depending on the composition of the CO-containing substrate, the CO-containing substrate may be provided directly to a fermentation process or may be further modified to include an appropriate H₂ to CO molar ratio. In one aspect, CO-containing substrate provided to the fermentor has an H₂ to CO molar ratio of about 0.2 or more, in another aspect, about 0.25 or more, and in another aspect, about 0.5 or more. In another aspect, CO-containing substrate provided to the fermentor may include about 40 mole percent or more CO plus H₂ and about 30 mole percent or less CO, in another aspect, about 50 mole percent or more CO plus H₂ and about 35 mole percent or less CO, and in another aspect, about 80 mole percent or more CO plus H₂ and about 20 mole percent or less CO.

In one aspect, the CO-containing substrate mainly includes CO and H₂. In this aspect, the CO-containing substrate will contain at least about 10 mole % CO, in one aspect, at least about 20 mole %, in one aspect, about 10 to about 100 mole %, in another aspect, about 20 to about 100 mole % CO, in another aspect, about 30 to about 90 mole % CO, in another aspect, about 40 to about 80 mole % CO, and in another aspect, about 50 to about 70 mole % CO. The CO-containing substrate will have a CO/CO₂ ratio of at least about 0.75, in another aspect, at least about 1.0, and in another aspect, at least about 1.5.

In one aspect, a gas separator is configured to substantially separate at least one portion of the gas stream, wherein the portion includes one or more components. For example, the gas separator may separate CO₂ from a gas stream comprising the following components: CO, CO₂, H₂, wherein the CO₂ may be passed to a CO₂ remover and the remainder of the gas stream (comprising CO and H₂) may be passed to a bioreactor. Any gas separator known in the art may be utilized. In this aspect, syngas provided to the fermentor will have about 10 mole % or less CO₂, in another aspect, about 1 mole % or less CO₂, and in another aspect, about 0.1 mole % or less CO₂.

Certain gas streams may include a high concentration of CO and low concentrations of H₂. In one aspect, it may be desirable to optimize the composition of the substrate stream in order to achieve higher efficiency of alcohol production and/or overall carbon capture. For example, the concentration of H₂ in the substrate stream may be increased before the stream is passed to the bioreactor.

According to particular aspects of the invention, streams from two or more sources can be combined and/or blended to produce a desirable and/or optimized substrate stream. For example, a stream comprising a high concentration of CO, such as the exhaust from a steel mill converter, can be combined with a stream comprising high concentrations of H₂, such as the off-gas from a steel mill coke oven.

Depending on the composition of the gaseous CO-containing substrate, it may also be desirable to treat it to remove any undesired impurities, such as dust particles before introducing it to the fermentation. For example, the gaseous substrate may be filtered or scrubbed using known methods.

### Bioreactor Design and Operation

Descriptions of fermentor designs are described in U.S. Ser. Nos. 13/471,827 and 13/471,858, both filed May 15, 2012, and U.S. Ser. No. 13/473,167, filed May 16, 2012, all of which are incorporated herein by reference.

In accordance with one aspect, the fermentation process is started by addition of medium to the reactor vessel. Some examples of medium compositions are described in U.S. Ser. Nos. 61/650,098 and 61/650,093, filed May 22, 2012, and in U.S. Pat. No. 7,285,402, filed Jul. 23, 2001, all of which are incorporated herein by reference. The medium may be sterilized to remove undesirable microorganisms and the reactor is inoculated with the desired microorganisms. Sterilization may not always be required.

In one aspect, the microorganisms utilized include acetogenic bacteria. Examples of useful acetogenic bacteria include those of the genus Clostridium, such as strains of Clostridium ljungdahlii, including those described in WO 2000/68407, EP 117309, U.S. Pat. Nos. 5,173,429, 5,593,886 and 6,368,819, WO 1998/00558 and WO 2002/08438, strains of Clostridium autoethanogenum (DSM 10061 and DSM 19630 of DSMZ, Germany) including those described in WO 2007/117157 and WO 2009/151342 and Clostridium ragsdalei (P11, ATCC BAA-622) and Alkalibaculum bacchi (CP11, ATCC BAA-1772) including those described respectively in U.S. Pat. No. 7,704,723 and “Biofuels and Bioproducts from Biomass-Generated Synthesis Gas”, Hasan Atiyeh, presented in Oklahoma EPSCoR Annual State Conference, Apr. 29, 2010 and Clostridium carboxidivorans (ATCC PTA-7827) described in U.S. Patent Application No. 2007/0276447. Other suitable microorganisms includes those of the genus Moorella, including Moorella sp. HUC22-1, and those of the genus Carboxydothermus. Each of these references is incorporated herein by reference. Mixed cultures of two or more microorganisms may be used.

Some examples of useful bacteria include Acetogenium kivui, Acetoanaerobium noterae, Acetobacterium woodii, Alkalibaculum bacchi CP11 (ATCC BAA-1772), Blautia producta, Butyribacterium methylotrophicum, Caldanaerobacter subterraneous, Caldanaerobacter subterraneous pacificus, Carboxydothermus hydrogenoformans, Clostridium aceticum, Clostridium acetobutylicum, Clostridium acetobutylicum P262 (DSM 19630 of DSMZ Germany), Clostridium autoethanogenum (DSM 19630 of DSMZ Germany), Clostridium autoethanogenum (DSM 10061 of DSMZ Germany), Clostridium autoethanogenum (DSM 23693 of DSMZ Germany), Clostridium autoethanogenum (DSM 24138 of DSMZ Germany), Clostridium carboxidivorans P7 (ATCC PTA-7827), Clostridium coskatii (ATCC PTA-10522), Clostridium drakei, Clostridium ljungdahlii PETC (ATCC 49587), Clostridium ljungdahlii ER12 (ATCC 55380), Clostridium ljungdahlii C-01 (ATCC 55988), Clostridium ljungdahlii O-52 (ATCC 55889), Clostridium magnum, Clostridium pasteurianum (DSM 525 of DSMZ Germany), Clostridium ragsdali P11 (ATCC BAA-622), Clostridium scatologenes, Clostridium thermoaceticum, Clostridium ultunense, Desulfotomaculum kuznetsovii, Eubacterium limosum, Geobacter sulfurreducens, Methanosarcina acetivorans, Methanosarcina barkeri, Morrella thermoacetica, Morrella thermoautotrophica, Oxobacter pfennigii, Peptostreptococcus productus, Ruminococcus productus, Thermoanaerobacter kivui, and mixtures thereof.

The fermentation should desirably be carried out under appropriate conditions for the desired fermentation to occur (e.g. CO-to-ethanol). Reaction conditions that should be considered include pressure, temperature, gas flow rate, liquid flow rate, media pH, media redox potential, agitation rate (if using a continuous stirred tank reactor), inoculum level, maximum gas substrate concentrations to ensure that CO in the liquid phase does not become limiting, and maximum product concentrations to avoid product inhibition.

The methods of the invention can be used to sustain the viability of a microbial culture, wherein the microbial culture is limited in CO, such that the rate of transfer of CO into solution is less than the uptake rate of the culture. Such situations may arise when a substrate comprising CO is not continuously provided to the microbial culture; the mass transfer rate is low; or there is insufficient CO in a substrate stream to sustain culture vitality at optimum temperature. In such embodiments, the microbial culture will rapidly deplete the CO dissolved in the liquid nutrient medium and become substrate limited as further substrate cannot be provided fast enough.

Startup:

Upon inoculation, an initial feed gas supply rate is established effective for supplying the initial population of microorganisms. Effluent gas is analyzed to determine the content of the effluent gas. Results of gas analysis are used to control feed gas rates. In this aspect, the process provides a calculated CO concentration to initial cell density ratio of about 0.5 to about 0.9, in another aspect, about 0.6 to about 0.8, in another aspect, about 0.5 to about 0.7, and in another aspect, about 0.5 to about 0.6.

In another aspect, a fermentation process includes providing syngas to a fermentation medium in an amount effective for providing an initial calculated CO concentration in the fermentation medium of about 0.15 mM to about 0.70 mM, in another aspect, about 0.15 mM to about 0.50 mM, in another aspect, about 0.15 mM to about 0.35 mM, in another aspect, about 0.20 mM to about 0.30 mM, and in another aspect, about 0.23 mM to about 0.27 mM. The process is effective for increasing cell density as compared to a starting cell density.

Post-Startup:

Upon reaching desired levels, liquid phase and cellular material is withdrawn from the reactor and replenished with medium. The process is effective for increasing cell density to about 2.0 grams/liter or more, in another aspect, about 2 to about 30 grams/liter, in another aspect, about 2 to about 25 grams/liter, in another aspect, about 2 to about 20 grams/liter, in another aspect, about 2 to about 10 grams/liter, in another aspect, about 2 to about 8 grams/liter, in another aspect, about 3 to about 30 grams/liter, in another aspect, about 3 to about 6 grams/liter, and in another aspect, about 4 to about 5 grams/liter.

Fermentations of CO-containing substrates conducted in bioreactors with medium and acetogenic bacteria as described herein are effective for providing a STY (space time yield) of at least about 10 g total alcohol/(L·day). Possible STY values include about 10 g total alcohol/(L·day) to about 200 g total alcohol/(L·day), in another aspect, about 10 g total alcohol/(L·day) to about 160 g total alcohol/(L·day), in another aspect, about 10 g total alcohol/(L·day) to about 120 g total alcohol/(L·day), in another aspect, about 10 g total alcohol/(L·day) to about 80 g total alcohol/(L·day), in another aspect, about 20 g total alcohol/(L·day) to about 140 g total alcohol/(L·day), in another aspect, about 20 g total alcohol/(L·day) to about 100 g total alcohol/(L·day), in another aspect, about 40 g total alcohol/(L·day) to about 140 g total alcohol/(L·day), and in another aspect, about 40 g total alcohol/(L·day) to about 100 g total alcohol/(L·day).

In another aspect, fermentations of CO-containing substrates conducted in bioreactors with medium and acetogenic bacteria as described herein are effective for providing a STY (space time yield) of at least about 10 g ethanol/(L·day). Possible STY values include about 10 g ethanol/(L·day) to about 200 g ethanol/(L·day), in another aspect, about 10 g ethanol/(L·day) to about 160 g ethanol/(L·day), in another aspect, about 10 g ethanol/(L·day) to about 120 g ethanol/(L·day), in another aspect, about 10 g ethanol/(L·day) to about 80 g ethanol/(L·day), in another aspect, about 20 g ethanol/(L·day) to about 140 g ethanol/(L·day), in another aspect, about 20 g ethanol/(L·day) to about 100 g ethanol/(L·day), in another aspect, about 40 g ethanol/(L·day) to about 140 g ethanol/(L·day), and in another aspect, about 40 g ethanol/(L·day) to about 100 g ethanol/(L·day).

### The Distillation Process

In one aspect, a process for producing an alcohol product composition includes providing a permeate to a distillation tower. Permeate may be provided from the fermentation process as described herein. In this aspect, permeate may include from about 1 to about 5 weight percent ethanol, in another aspect, about 1 to about 4 weight percent ethanol, in another aspect, about 1 to about 3 weight percent ethanol, in another aspect, about 2 to about 3 weight percent ethanol, in another aspect, about 2 to about 4 weight percent ethanol, and in another aspect, about 2 to about 5 weight percent ethanol.

The present process utilizes a continuous distillation process. Industrial distillation is typically performed in large, vertical cylindrical columns (commonly referred to as distillation columns, distillation towers or fractionators with diameters ranging from about 65 centimetres to 11 meters and heights ranging from about 6 meters to 60 meters or more.

To provide for the intimate mixing of the upward flowing vapor and downward flowing liquid in distillation columns, the columns usually contain a series of horizontal distillation trays or plates. The distillation trays or plates are typically separated by about 45 to 75 centimetres of vertical distance. However, in some aspects columns may be used which are designed to use beds of packing media rather than trays or plates.

In the present process, know distillation towers may be utilized and run generally according to manufacturer's recommendations. Some examples of commercially available distillation towers include for example, Vogelbush (Austria).

Permeate is provided to the distillation tower and an ethanol draw-off composition is removed from the distillation tower. A side-draw from the distillation tower is removed to provide a side-draw composition. In this aspect, the ethanol draw-off and side-draw composition are combined prior to dehydration. In an alternative aspect, ethanol and fusel oil may be removed from the distillation column together and provided to dehydration.

In another aspect, dehydration may be provided by any known process and equipment. For example, a mole sieve may be utilized to provide dehydration.

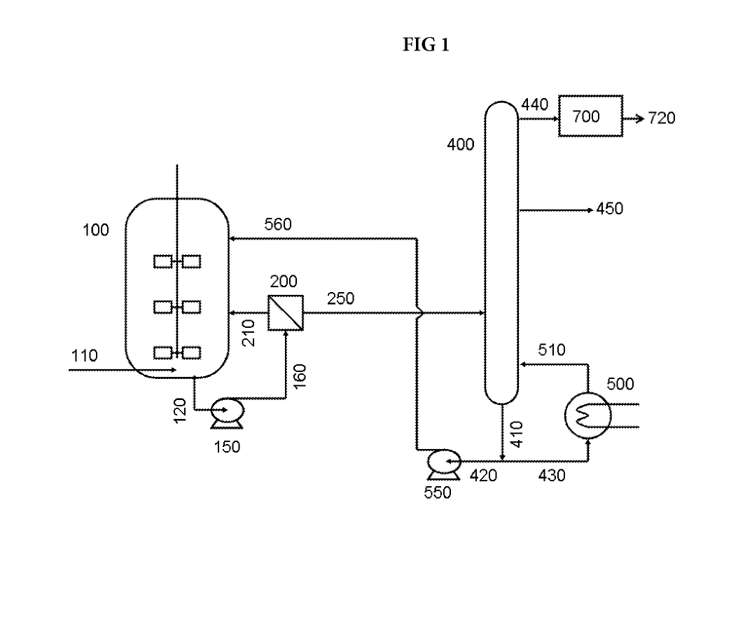

FIG. 1 illustrates a process and system for fermentation of syngas and production of an alcohol product. Syngas enters reactor vessel 100 through a syngas inlet 110. Medium and cells and are drawn out through medium outlet 120 and supplied to a cell separation filter 200 through filter supply 160 using a medium recirculation pump 150. The cell separation filter 200 provides concentrated cells and permeate. The reactor vessel 100 receives concentrated cells through cell recycle line 210 and a distillation column 400 receives permeate through a permeate supply 250. The distillation column 400 provides ethanol/water 440 and a reduced ethanol aqueous stream 410. A molecular sieve/dryer 700 may receive the ethanol/water 440 and provide ethanol product 720. A reboiler 500 receives a portion of the reduced ethanol aqueous stream 410 through a reboiler supply line 430. The reboiler 500 provides a preheated reduced ethanol aqueous stream 510. An aqueous stream recirculation pump 550 receives the reduced ethanol aqueous stream through aqueous supply line 420. The aqueous stream recirculation pump 550 provides the reduced ethanol aqueous stream back to the reactor vessel 100 through a reduced ethanol aqueous stream supply line 560.

In another aspect, a fusel oil may be removed from the distillation column 400 at side draw 450. As used herein, “fusel oil” may include amyl alcohol, propanol, butanol, fatty acids, esters, and mixtures thereof.

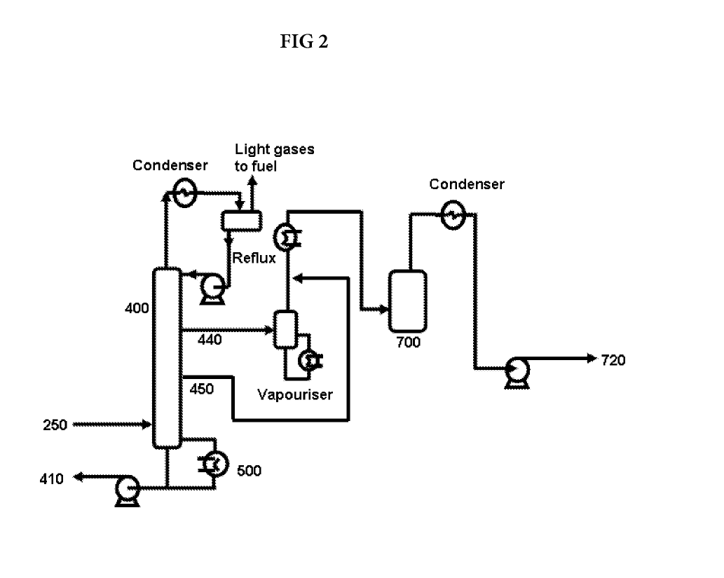

FIG. 2 generally illustrates a distillation process. A permeate supply 250 enters a distillation column 400. The distillation column 400 provides ethanol/water 440 and a reduced ethanol aqueous stream 410. A fusel oil may be removed from the distillation column 400 at side draw 450. The ethanol/water 440 and fusel oil side draw 450 may be recombined prior to entering the mole sieve/dryer 700. The process produces an alcohol product 720.

### The Alcohol Product

The alcohol product may include 92 weight percent or more ethanol, in another aspect, 93 weight percent or more ethanol, in another aspect, 94 weight percent or more ethanol, in another aspect, 95 weight percent or more ethanol, in another aspect, 96 weight percent or more ethanol, in another aspect, 97 weight percent or more ethanol, in another aspect, 98 weight percent or more ethanol, and in another aspect, 99 weight percent or more ethanol.

The alcohol product may include about 0.5 to about 8 weight percent organic composition, in another aspect, about 0.5 to about 7 weight percent organic composition, in another aspect, about 0.5 to about 6 weight percent organic composition, in another aspect, about 0.5 to about 5 weight percent organic composition, in another aspect, about 0.5 to about 4 weight percent organic composition, in another aspect, about 0.5 to about 3 weight percent organic composition, in another aspect, about 0.5 to about 2 weight percent organic composition, and in another aspect, about 0.5 to about 1.5 weight percent organic composition. In this aspect, the organic composition may include n-butanol, isobutanol, pentanol, hexanol, propanol, methanol, ethyl acetate, fatty acids, esters and mixtures thereof. In one aspect, the organic composition is substantially n-butanol (>98 weight percent). In this aspect, the alcohol product includes about 0.5 to about 6 weight percent n-butanol, in another aspect, about 0.5 to about 5 weight percent n-butanol, in another aspect, about 0.5 to about 4 weight percent n-butanol, in another aspect, about 0.5 to about 3 weight percent n-butanol, in another aspect, about 0.5 to about 2 weight percent n-butanol, and in another aspect, about 0.5 to about 1.5 weight percent n-butanol.

The alcohol product may include about 1 weight percent or less water, in another aspect, about 0.9 weight percent or less water, in another aspect, about 0.8 weight percent or less water, in another aspect, about 0.7 weight percent or less water, in another aspect, about 0.6 weight percent or less water, and in another aspect, about 0.5 weight percent or less water.

The alcohol product may include about 0.5 weight percent or less organic acid, in another aspect, about 0.4 weight percent or less organic acid, in another aspect, about 0.3 weight percent or less organic acid, and in another aspect, about 0.2 weight percent or less organic acid. In this aspect, the organic acid may include organic acids selected from the group consisting of acetic acid, butyric acid, maleic acid, malonic acid, malic acid, ethanoic acid, propionic acid, succinic acid, oxalic acid, lactic acid, fumaric acid, glutaric acid, formic acid, citric acid, uric acid, and mixtures thereof. In one aspect, the alcohol product has a pHe of about 5 to about 9, in another aspect, about 6.5 to about 9, in another aspect, about 6 to about 8, and in another aspect, about 6.5 to about 7. Determination of pHe may be in accordance with ASTM D6423 which is incorporated herein by reference.

The present alcohol composition may be used in a variety of applications including applications as fuels, solvents, chemical feedstocks, pharmaceutical products, cleansers, sanitizers, hydrogenation transport or consumption. In fuel applications, the finished alcohol composition may be blended with gasoline for motor vehicles such as automobiles, boats and small piston engine aircraft. In non-fuel applications, the finished alcohol composition may be used as a solvent for toiletry and cosmetic preparations, detergents, disinfectants, coatings, inks, and pharmaceuticals. The finished alcohol composition may also be used as a processing solvent in manufacturing processes for medicinal products, food preparations, dyes, photochemicals and latex processing.

The finished alcohol composition may also be used as a chemical feedstock to make other chemicals such as vinegar, ethyl acrylate, ethyl acetate, ethylene, glycol ethers, ethylamines, aldehydes, and higher alcohols, especially butanol. In the production of ethyl acetate, the finished alcohol composition may be esterified with acetic acid. In another application, the finished alcohol composition may be dehydrated to produce ethylene.

In one aspect, the alcohol composition may be blended with one or more hydrocarbons, such as for example gasoline. In this aspect, the alcohol composition may include from about 0.5 to about 10 weight percent gasoline, in another aspect, about 0.5 to about 8 weight percent gasoline, in another aspect, about 0.5 to about 6 weight percent gasoline, in another aspect, about 0.5 to about 4 weight percent gasoline, in another aspect, about 0.5 to about 2, and in another aspect, about 0.5 to about 1. In another aspect, the alcohol composition includes less than about 0.5 weight percent gasoline.

### EXAMPLES

### Example 1

### Distillation Column Operation

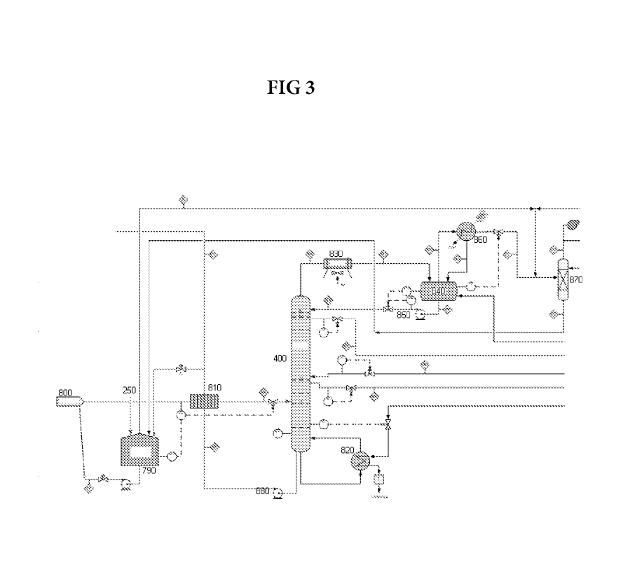

As illustrated in FIG. 3, a permeate feed 250 was provided to a distillation column 400. The permeate feed 250 contained approximately 3% ethanol and 97% water. The permeate feed 250 was pumped from a feed tank through an alcohol vapor/feed exchanger 800 where it was preheated from 100° F. to 111° F. with condensing product alcohol vapor. Before entering the distillation column 400, the feed was preheated further to 220° F. in a distillation column feed heater 810 with rectifying column bottoms.

Inside the distillation column 400, the alcohol was concentrated to near its azeotropic point at the top of the column and water containing less than 100 ppm alcohol was discharged from the bottom. The heat required to drive the column was a 50 psig steam supplied to a thermo-siphon reboiler 820 from a low pressure steam header. Steam condensate from a rectifier reboiler was returned to a condensate flash drum.

A purge recycle stream from the molecular sieve unit (MSU) was added to Tray 23 for the recovery of alcohol. A side draw of fusel oil was withdrawn from Tray 22 in vapor form and routed through the feed superheater directly to the MSU for dehydration. A mist eliminator knock-out pot allows any liquid droplets to collect and drain back into a rectifying column. The majority of the alcohol is withdrawn as liquid from Tray 57. The pressure in the distillation column pushes the liquid alcohol into a MSU vaporizer heater where it is vaporized before being sent to the MSU for dehydration. Collecting the alcohol as a liquid from the column prevents non-condensibles and contaminants from being sent to the MSU. Instead, the non-condensibles and contaminants exit the column with the overhead vapors and were vented through a vent scrubber.

The liquid flow of alcohol from the column is determined by temperatures in the column and adjusted to maintain the material balance in the system. Overhead vapors from the distillation column 400 are withdrawn at the top of the column and condensed inside the tubes of an overhead condenser 830 which uses ambient air cooling. The condensed vapors are collected in the reflux drum 840, from where they are returned to the column 400 with a distillation column reflux pump 850.

Non-condensables from the reflux drum 840 were routed to the reflux drum vent condenser 860. This vent stream contained low boiling temperature byproducts and ethanol. The ethanol was condensed using cooling water and flows back to the reflux drum 840. The remaining non-condensables from the reflux drum vent condenser 860 were sent to a vent scrubber 870, where they were scrubbed free of any remaining alcohol using water. The alcohol-free non-condensables were then sent to a boiler or flare using a vent scrubber blower. The distillation column 400 top pressure is maintained with a control valve on a vent line from the reflux drum vent condenser 840. The bottoms of the column 400 were pumped using a bottoms pump 880 through the column feed heater 810, where they are cooled while preheating the column feed. From there, the column bottoms were sent to 2^{nd} growth and production fermenters.

The alcohol product composition produced in accordance with the present process had the following components.

| Method | Test | Result | Units |
|---|---|---|---|
| ASTM D2699 | Procedure Used | Bracketing-EFL | |
| | Engine Room Barometric Pressure | 29.97 | in Hg |
| | Intake Air Temperature | 126 | ° F. |
| | Research O.N. | 105 | |
| ASTM D2700 | Procedure Used | Compression Ratio | |
| | Engine Room Barometric Pressure | 29.97 | in Hg |
| | Mixture Temperature | 300 | ° F. |
| | Motor O.N. | 89.2 | |
| ASTM D4814- | Antiknock Index (Octane Rating) | 97.1 | |
| X1.4 | | | |
| ASTM D5191 | Dry Vapor Pressure Equivalent, | 2.97 | psi |
| | EPA | | |
| | Container Size | 1-L | |
| | Observed Condition | Sample is not hazy | |
| ASTM D4327 | Chloride | <1.0 | ppm |
| | | | (mg/ |
| | | | kg) |
| | Bromide | <1.0 | ppm |
| | | | (mg/ |
| | | | kg) |
| | Fluorine | <1.0 | ppm |
| | | | (mg/ |
| | | | kg) |
| | Sulfate | <1.0 | ppm |
| | | | (mg/ |
| | | | kg) |
| | Nitrate | <1.0 | ppm |
| | | | (mg/ |
| | | | kg) |
| | Fluoride | <1.0 | ppm |
| | | | (mg/ |
| | | | kg) |
| EN 15721 | Procedure A | | |
| | 1-Propanol | 0.173 | Wt % |
| | 1-Butanol | 0.948 | Wt % |
| | 2-Butanol | 0.017 | Wt % |
| | 2-Methyl-1-Propanol | 0.272 | Wt % |
| | 2-Methyl-1-Butanol | 0.022 | Wt % |
| | 3-Methyl-1-Butanol | 0.009 | Wt % |
| | Other Identified Higher Alcohol | 0.005 | Wt % |
| | Impurities | | |
| | Higher Alcohols | 1.446 | Wt % |
| | Methanol | 0.006 | Wt % |
| | Ethyl-Ethanoate (=Ethylacetate) | 0.015 | Wt % |
| | Ethanal (=Acetic Aldehyde) | 0.004 | Wt % |
| | 1,1-diethoxyethane (=Acetal) | 0.001 | Wt % |
| | Other Identified Oxygenated | <0.001 | Wt % |
| | Compounds | | |
| | Unidentified Compounds | 0.04 | Wt % |
| | Impurities | 0.060 | Wt % |
| | Ethanol, including Higher Alcohols | 99.934 | Wt % |
| EN 15938 | Conductivity at 25° C. | 1.02 | μS/cm |
| ASTM D5501 | Ethanol Content | 98.23 | Wt % |
| | Methanol Content | 0.01 | Wt % |
| BY DIFFERENCE | Estimated Denaturant Content | 1.40 | Vol % |

While the invention herein disclosed has been described by means of specific embodiments, examples and applications thereof, numerous modifications and variations could be made thereto by those skilled in the art without departing from the scope of the invention set forth in the claims.

What is claimed is:

1. An alcohol product composition comprising:
about 92 weight percent or more ethanol;
about 0.5 to about 8 weight percent organic composition;
about 1 weight percent or less water; and
about 0.5 weight percent or less organic acid.

2. The composition of claim 1 wherein the organic composition is selected from the group consisting of n-butanol, isobutanol, pentanol, hexanol, propanol, methanol, ethyl acetate, fatty acids, esters and mixtures thereof.

3. The composition of claim 1 wherein the organic composition further includes one or more hydrocarbons.

4. The composition of claim 3 wherein the one or more hydrocarbons includes gasoline.

5. The composition of claim 2 wherein the organic composition is substantially n-butanol.

6. The composition of claim 5 wherein the alcohol product composition includes about 0.5 weight percent to about 6 weight percent n-butanol.

7. The composition of claim 1 wherein the organic acid is selected from the group consisting of acetic acid, butyric acid, maleic acid, malonic acid, malic acid, ethanoic acid, propionic acid, succinic acid, oxalic acid, lactic acid, fumaric acid, glutaric acid, formic acid, citric acid, uric acid, and mixtures thereof.

8. The composition of claim 1 wherein the alcohol composition has less than about 0.5 weight percent gasoline.

9. A process for producing an alcohol product composition, the process comprising:
providing a permeate to a distillation tower;
removing an ethanol draw-off composition from the distillation tower;
removing a side draw from the distillation tower to provide side-draw composition;
combining the ethanol draw-off composition and side-draw composition to provide a alcohol composition having more than 1 weight percent water; and
dehydrating the alcohol composition to provide an alcohol product composition having at about 92 weight % or more ethanol, about 1 weight % or less water, about 0.5 to about 8 weight percent organic composition, and about 0.5 weight percent or less organic acid.

10. The process of claim 9 wherein the permeate includes from about 1 to about 5 weight percent ethanol.

11. The process of claim 10 wherein the permeate is provided from a fermentation of a CO-containing gaseous substrate.

12. The process of claim 11 wherein the fermentation is effective for providing a STY of at least about 10 g total alcohol/(L·day).

13. The process of claim 11 wherein the fermentation is effective for providing a STY of at least about 10 g ethanol/(L·day).

14. The process of claim 9 wherein the organic composition is selected from the group consisting of n-butanol, isobutanol, pentanol, hexanol, propanol, methanol, ethyl acetate, fatty acids, esters and mixtures thereof.

15. The process of claim 14 wherein the organic composition is n-butanol.

16. The process of claim 15 wherein the alcohol product composition includes about 0.5 weight percent to about 6 weight percent n-butanol.

17. The process of claim 9 wherein the organic acid is selected from the group consisting of acetic acid, butyric acid, maleic acid, malonic acid, malic acid, propionic acid, succinic acid, oxalic acid, lactic acid, fumaric acid, glutaric acid, formic acid, citric acid, uric acid, and mixtures thereof.

18. A bioethanol product comprising:
about 92 weight percent or more ethanol;
about 0.5 to about 8 weight percent organic composition;
about 1 weight % or less water; and
about 0.5 weight percent or less organic acid, wherein the bioethanol product is produced by a process that includes
fermenting a CO-containing substrate and separating a permeate from the fermentation;
providing the permeate to a distillation tower;
removing an ethanol draw-off composition from the distillation tower;
removing a side draw from the distillation tower to provide side-draw composition; and
combining the ethanol draw-off composition and side-draw composition to provide a alcohol composition having more than 1 weight percent water; and dehydrating the alcohol composition to provide the bioethanol product.

19. The composition of claim 18 wherein the permeate includes from about 1 to about 5 weight percent ethanol.

20. The composition of claim 18 wherein the organic composition is selected from the group consisting of n-butanol, isobutanol, pentanol, hexanol, propanol, methanol, ethyl acetate, fatty acids, esters and mixtures thereof.

21. The composition of claim 20 wherein the organic composition is n-butanol.

22. The composition of claim 21 wherein the bioethanol product composition includes about 0.5 weight percent to about 6 weight percent n-butanol.

23. The composition of claim 18 wherein the organic acid is selected from the group consisting of acetic acid, butyric acid, maleic acid, malonic acid, malic acid, propionic acid, succinic acid, oxalic acid, lactic acid, fumaric acid, glutaric acid, formic acid, citric acid, uric acid, and mixtures thereof.

24. A process for producing an alcohol product composition, the process comprising:
fermenting a CO-containing gaseous substrate to produce a first alcohol composition;
purifying part or all of the first alcohol composition to produce a second alcohol product composition having about 92 weight percent or more ethanol; about 0.5 to about 8 weight percent organic composition; about 1 weight % or less water; and about 0.5 weight percent or less organic acid.

25. The process of claim 24 wherein purifying is selected from the group consisting of dehydration, filtration and mixtures thereof.

26. The process of claim 24 wherein the first alcohol composition includes from about 1 to about 5 weight percent ethanol.

27. The process of claim 24 wherein the first alcohol composition is provided from a fermentation of a CO-containing gaseous substrate.

28. The process of claim 27 wherein the fermentation is effective for providing a STY of at least about 10 g total alcohol/(L·day).

29. The process of claim 27 wherein the fermentation is effective for providing a STY of at least about 10 g ethanol/(L·day).

30. The process of claim 24 wherein the organic composition is selected from the group consisting of n-butanol, isobutanol, pentanol, hexanol, propanol, methanol, ethyl acetate, fatty acids, esters and mixtures thereof.

31. The process of claim 30 wherein the organic composition is n-butanol.

32. The process of claim 31 wherein the alcohol product composition includes about 0.5 weight percent to about 6 weight percent n-butanol.

33. The process of claim 24 wherein the organic acid is selected from the group consisting of acetic acid, butyric acid, maleic acid, malonic acid, malic acid, propionic acid, succinic acid, oxalic acid, lactic acid, fumaric acid, glutaric acid, formic acid, citric acid, uric acid, and mixtures thereof.
